(19) United States
(12) Patent Application Publication
(10) Pub. No.: US 2010/0016666 A1
(43) Pub. Date: Jan. 21, 2010

(54) SURGICAL INSTRUMENT POSITION CONTROL APPARATUS FOR ENDOSCOPE

(75) Inventor: Jun HASEGAWA, Hino-shi (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS MEDICAL SYSTEMS CORP., Tokyo (JP)

(21) Appl. No.: 12/568,057

(22) Filed: Sep. 28, 2009

Related U.S. Application Data
(63) Continuation of application No. PCT/JP2008/053087, filed on Feb. 22, 2008.

(30) Foreign Application Priority Data
Mar. 29, 2007 (JP) 2007-089715

(51) Int. Cl. A61B 1/00; A61B 1/04

(52) U.S. Cl. 600/118

(57) ABSTRACT

An endoscopic system includes a surgical instrument position control apparatus. The surgical instrument position control apparatus estimates motion of an endoscope main body with respect to a target site, based on an image picked up in conjunction with movement of an endoscope. To determine the moving amount of the surgical instrument, the surgical instrument position control apparatus calculates the amounts of operations, such as bending, rotation, and forward and backward movements, of each joint of a surgical arm section which amounts are required to move the surgical instrument from a position taken by the surgical instrument after movement of the endoscope to a surgical procedure position. The surgical instrument position control apparatus thus bends each joint to move the surgical section to the original surgical procedure position so as to hold the position of the surgical instrument every time the endoscope is moved.

### CROSS REFERENCE TO RELATED APPLICATIONS

This is a Continuation application of PCT Application No. PCT/JP2008/053087, filed Feb. 22, 2008, which was published under PCT Article 21(2) in Japanese.

This application is based upon and claims the benefit of priority from prior Japanese Patent Application No. 2007-089715, filed Mar. 29, 2007, the entire contents of which are incorporated herein by reference.

### BACKGROUND OF THE INVENTION

1. Field of the Invention

The present invention relates to a surgical instrument position control apparatus that controls the position of a surgical instrument inserted through a forceps channel in an endoscope.

2. Description of the Related Art

An endoscope is commonly known as an instrument for observing a possible lesion or the like in the body cavity. The endoscope includes an image pickup section at the distal end portion of an insertion section of the endoscope which is inserted into the body cavity, or in the main body of the apparatus. The endoscope thus displays a desired observation target on a monitor as an image. The insertion section is flexible and includes a channel (forceps channel) penetrating the endoscope from a proximal end side to the distal end. A surgical instrument such as a pair of forceps or an electric scalpel is inserted through an insertion port of the forceps channel as required. Thus, a surgeon can perform various surgical procedures on the lesion or the like while observing the endoscopic image.

Conventionally, the surgeon holds a manipulation section with one hand to manually manipulate the manipulation section to appropriately bend a bending section of the insertion section while observing the lesion or the like. The surgeon simultaneously manipulates the surgical instrument with the other hand. In recent years, to reduce the burden of endoscope manipulation on the surgeon, electrically-operated endoscopes have been proposed as disclosed in, for example, Japanese Patent No. 3007715. Furthermore, for surgical instruments, in order to not only reduce the burden of manipulation on the surgeon but also reduce operation time and thus burdens on a patient, electrically-operated robotized surgical instruments have been proposed as disclosed in, for example, Jpn. Pat. Appln. KOKAI Publication No. 2003-127076.

As described above, the surgical instrument is normally inserted from the insertion port of the forceps channel to a forceps port at the distal end. The pair of forceps or electric scalpel projects from the distal end of the surgical instrument. The surgical instrument can be moved forward and backward through the forceps channel and moves integrally with the distal end portion of the endoscope. That is, as the distal end portion of the endoscope moves, the surgical instrument equivalently moves.

Namely, during operation, when the surgeon changes an observation visual field or the endoscope is moved by biological motion (peristaltic motion, a breath, the heart, or the like) in the body cavity of patient, the surgical instrument inserted from the proximal end side to distal end of the endoscope is integrally moved. The surgeon thus needs to perform an operation of returning the distal end of the surgical instrument to the original position of the surgical procedure.

### BRIEF SUMMARY OF THE INVENTION

An surgical instrument position control apparatus according to an aspect of the present invention comprises a surgical instrument position control apparatus for an endoscope an endoscope allowing a biomedical tissue in the body cavity to be observed comprising, the surgical instrument having a surgical section which is movable forward and backward through an insertion section of the endoscope and which allows the biomedical tissue to be operated on, and a movable section the surgical section to bend and move forward and backward, a driving mechanism bending and moving forward and backward the movable section of the surgical section to move a position of the surgical section, an estimation section estimating a moving direction and a moving amount of the surgical section in conjunction with movement of a distal end of the insertion section, and a control section drivingly controlling the driving mechanism based on the moving direction and amount of the surgical section so that the surgical section is returned to a position taken by the surgical section before the endoscope is moved.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1 is a diagram showing the general configuration of an endoscopic system in which a surgical instrument position control apparatus according to a first embodiment is mounted;

FIG. 2 is a flowchart illustrating a moving process according to the first embodiment;

FIG. 3A is a diagram illustrating estimation of motion of the distal end of the endoscope;

FIG. 3B is a diagram showing the external configuration of the distal end of the endoscope;

FIG. 3C is a diagram showing the position of a surgical section at the distal end of the endoscope during movement of the surgical section;

FIG. 4 is a diagram showing the general configuration of an endoscopic system in which a surgical instrument position control apparatus according to a second embodiment is mounted;

FIG. 5A is a diagram illustrating estimation of movement of the surgical section based on the estimated motion of the distal end of the endoscope in an endoscopic system according to a third embodiment;

FIG. 5B is a diagram showing the surgical procedure position of the endoscope in the endoscopic system according to the third embodiment;

FIG. 5C is a diagram showing the surgical procedure position of the endoscope observed after movement of the endoscope, in the endoscopic system according to the third embodiment;

FIG. 6 is a diagram showing the general configuration of an endoscopic system in which a surgical instrument position control apparatus according to a fourth embodiment is mounted; and

FIG. 7 is a flowchart illustrating an endoscopic system moving process according to a fifth embodiment.

### DETAILED DESCRIPTION OF THE INVENTION

Embodiments of the present invention will be described below with reference to the drawings.

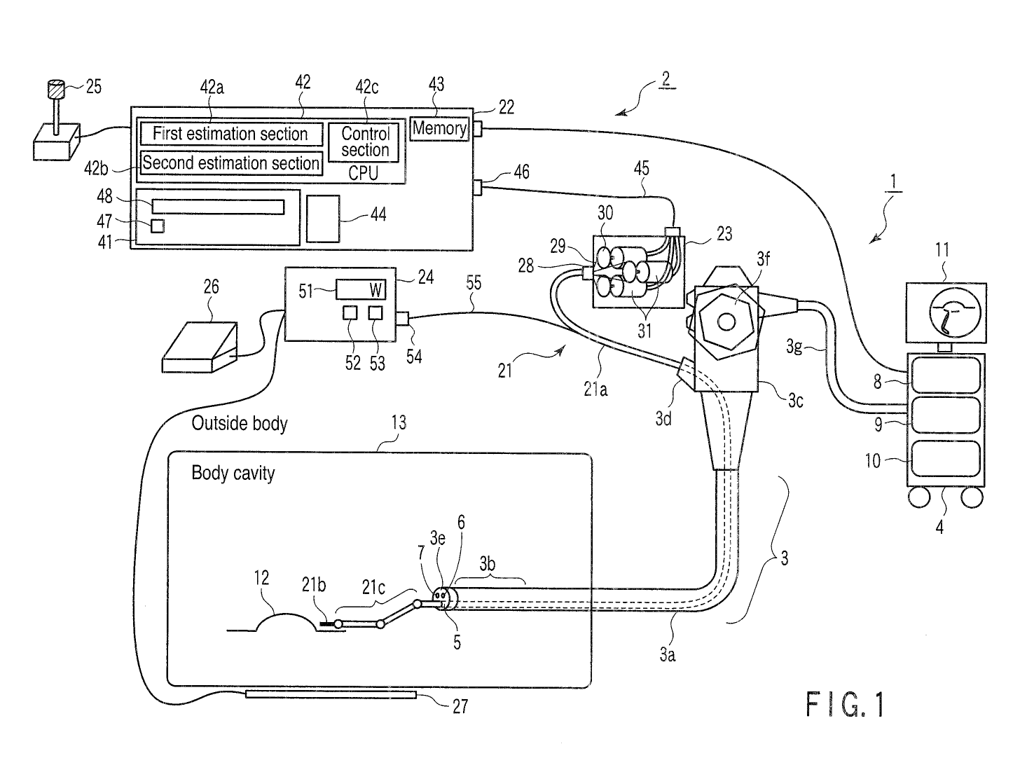

FIG. 1 is a diagram showing the general configuration of an endoscopic system in which a surgical instrument position control apparatus according to a first embodiment is mounted.

The endoscopic system is roughly composed of an endoscope 1 and a surgical instrument position control apparatus 2. The present invention is applicable to an electronic endoscope including an image pickup element at the distal end of an insertion section and an endoscope that takes an optical image guided through a fiber scope. In the embodiments described below, an electronic endoscope will be described by way of example.

An endoscope 1 is composed of an endoscope main body 3 and an apparatus main body 4. The endoscope main body 3 is composed of an insertion section 3a inserted into the body cavity, and a manipulation section 3c used to bend a bending section 3b provided on the distal end side of the insertion section 3a. The insertion section 3a includes, for example, a hole penetrating the insertion section 3a from an insertion port 3d that is open on the proximal end side, to a distal end 3e, that is, what is called a forceps port 5 for a forceps channel, and a light guide fiber 6 through which illumination light illuminating an observation visual field is propagated. Moreover, the distal end 3e has an image pickup section 7 including an image pickup element such as a CCD and an optical system. Image data on a lesion or the like obtained by the image pickup section 7 is transmitted, through the manipulation section 3c, to an image processing section 8 connected to the image pickup section 7, described below, of the apparatus main body 4 via a cable (the light guide fiber, an image signal line and a control signal line, and the like) 3g. The manipulation section 3c includes a manipulation dial 3f manipulated by the surgeon to bend the bending section 3b to bring a desired observation target (a lesion) 12 into an observation visual field (or an image pickup visual field).

The apparatus main body 4 is composed of an image processing section 8 executing various types of image processing and data processing on image data obtained by the image pickup section 7, a light source section 9 generating illumination light with which the observation visual field is irradiated through an illumination light window 6 via a light guide fiber, a control section 10 controlling the whole endoscopic system and executing arithmetic processing and the like, and a monitor 11 displaying picked-up images, data on the images, the status of the apparatus, operational instructions, and the like.

The surgical instrument position control apparatus 2 is composed of a surgical instrument 21 to which, for example, a high-frequency electric scalpel used to operate on the lesion 12 is attached, a surgical instrument control section 22 controlling the surgical instrument 21, a motor unit driving the surgical instrument 21 based on a control signal from the surgical instrument control section 22, and a power source device 24 supplying high-frequency power to the high-frequency electric scalpel, which is the surgical instrument 21. Moreover, the surgical instrument position control apparatus 2 includes a joystick 25 connected to the surgical instrument control section 22 and serving as an input device manually operated by the surgeon to specify the position and posture of the surgical instrument 21, a foot switch 26 connected to the power source device 24 and operated by the surgeon's foot to specify that high-frequency power be supplied to the high-frequency electric scalpel, and a counter electrode plate 27 connected to the power source device 24 and stuck to the body surface of a patient 13.

The surgical instrument position control apparatus 2 is a master slave-type electric surgical member enabling the surgical section to be moved to a desired position in accordance with manipulation of the joystick 25. If with the surgical instrument 21 according to the present embodiment set to be positionally controlled, the surgeon or the like manipulates the joystick 25, the instruction for the manipulation of the joystick 25 is given top priority.

The surgical instrument 21 is composed of a surgical instrument insertion section 21a inserted through the forceps channel and which is movable forward and backward, the surgical instrument insertion section 21a having a flexible property, a surgical section 21b, for example, the high-frequency electric scalpel, which is used to operate on the lesion 12, and a movable section (surgical arm section) 21c coupled to the surgical instrument insertion section 21a and the surgical section 21b and enabling the surgical section 21b to be three-dimensionally moved. The surgical arm section 21c is configured to three-dimensionally move the surgical section 21b. In the present embodiment, the surgical arm section 21c adopts, for example, an articulated structure (bending, axial rotation, and the like) having a combination of a plurality of joints and short rods. Any of various components, for example, piezoelectric elements shaped like cylinders, can be used as the movable section.

The proximal end side of the surgical instrument insertion section 21a is connected to a motor unit 23 via a connection section 28. The motor unit 23 is composed of wires 29 one end of which is connected to the joints of the surgical arm section 21c through the surgical instrument insertion section 21a, pulleys 30 coupled to the other ends of the respective wires 29, and motors 31 each including a rotating shaft on which the corresponding one of the pulleys 30 is fittingly installed. The motors 31 are individually drivingly controlled by the surgical instrument control section 22. In this configuration, the surgical instrument control section 22 performs driving control to rotate the motors 31 so that the joints of the surgical arm section 21c are bent by the traction force of the wires 29 wound around the pulleys 30. Furthermore, an actuator (not shown in the drawings) provided in the motor unit 23 and made up of a motor or the like moves forward and backward and rotates the surgical instrument insertion section 21 so as to rotate and move forward and backward the surgical section 21b.

The surgical instrument control section 22 is composed of a function control input section 41 to which instructions given using the joystick 25 and conditions and parameters for function control are input, a central processing unit (CPU) 42, a memory 43 to which images, communication data, and the like are saved, a motor driver 44 drivingly controlling the motors 31 in the motor unit 23, and a motor unit control section 46 connected to the motor unit 23 via a cable 45 for communication.

CPU 42 is roughly separated into a first estimation section 42a executing arithmetic processing of estimating the amount of motion (motion vector) of the endoscope based on image data obtained by the image pickup section 7 of the endoscope, a second estimation section 42b which, from the motion and the motion amount, calculates a movement direction and a movement amount to be provided to the surgical arm section 21c in order to allow the surgical section 21b to return to a surgical procedure position in the endoscope taken by the surgical section 21b before the movement, and a control section 42c controlling the components of the surgical instrument position control apparatus 2. The memory 43 stores images received by an image receiving section receiving images taken by the image pickup section 7 located at the distal end of the endoscope, the results of calculations by CPU 42, communication data, and the like. Moreover, the function control input section 41 includes a switch 47 used to turn on and off the function of fixing the distal end of the surgical section 21b at a particular position of an observation target, and a display 48 showing the status of the function.

According to the amount by which the surgeon has manipulated the joystick 25, the surgical instrument control section 22 transmits a control signal allowing the respective motors 31 to be driven, to the motor driver 44, to rotate the motors 31. An encoder (not shown in the drawings) is attached to each of the motors 31 to measure the rotational speed of the motor. The encoder generates and transmits a signal corresponding to the rotational speed, to the surgical instrument control section 22. Thus, the encoder performs feedback control on the motor 31.

The power source device 24 includes a display 51 displaying the status of power supply, an output wattage input panel 52, an output mode selection panel 53, and a power output terminal 54. The power output terminal 54 supplies high-frequency power output by a power source unit (not shown in the drawings) provided in the power source device 24, to a high-frequency electric scalpel through a cable 55. The cable 55 is inserted through the surgical instrument insertion section 21a together with the above-described wires 29 and connected to the high-frequency electric scalpel.

The endoscopic system in which the surgical instrument position control apparatus configured as described above is mounted estimates the motion of the bending section 3b of the endoscope with respect to the lesion 12, that is, the observation target, based on time-sequentially adjacent images. Based on the estimated motion, the endoscopic system determines the motion of the surgical section 21b located at the distal end of the surgical instrument. Based on the motion of the surgical section 21b, the system calculates the amounts of operations of the joints such as bending, rotation, and forward and backward movements which are required to return the surgical section 21b to the position taken by the surgical section 21b before the movement. The system thus moves the surgical section 21b to the position taken by the surgical section 21b before the movement. Every time the surgical section 21b moves, the movement process is repeatedly executed. In the embodiments of the present invention, the position taken by the surgical section 21b before the movement is not a single point expressed in three-dimensional coordinates but a general position determined with the vicinity of the point taken into account.

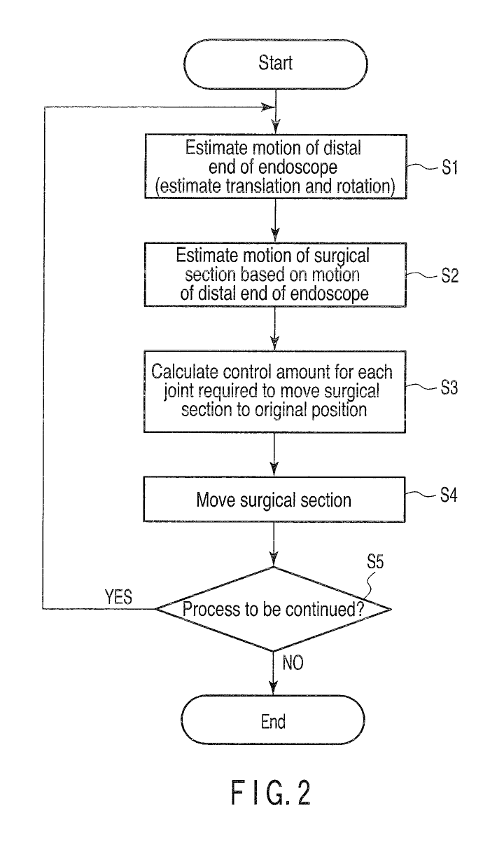

This moving process will be described in detail with reference to the flowchart shown in FIG. 2.

First, the surgical instrument control section 22 estimates the motion of the endoscope with respect to the target site based on time-sequentially adjacent image data obtained by the image pickup section 7 (step S1). Specifically, based on image data time-sequentially consecutively input by the endoscope, the surgical instrument control section 22 creates inter-image shift maps. In this technique, for example, shift maps are created for an optionally specified target site (for example, the lesion 12) in each screen obtained so that one inter-image shift map corresponds to estimation of one inter-image motion vector (a translation vector (h) and rotation matrix R: the translation vector is a unit vector). The motion vector, that is, the motion of the distal end of the endoscope, is estimated based on the shift map corresponding to each image. The translation vector and the rotation matrix are disclosed in, for example, Japanese Patent No. 3347385. Furthermore, a method of estimating the magnitude of a translation vector (H=kh) is disclosed in, for example, Jpn. Pat. Appln. KOKAI Publication No. 9-026547. The method is thus well known and will not be described in detail.

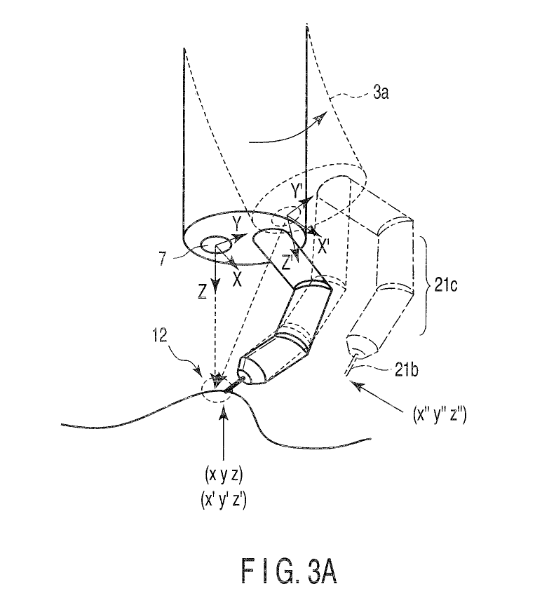

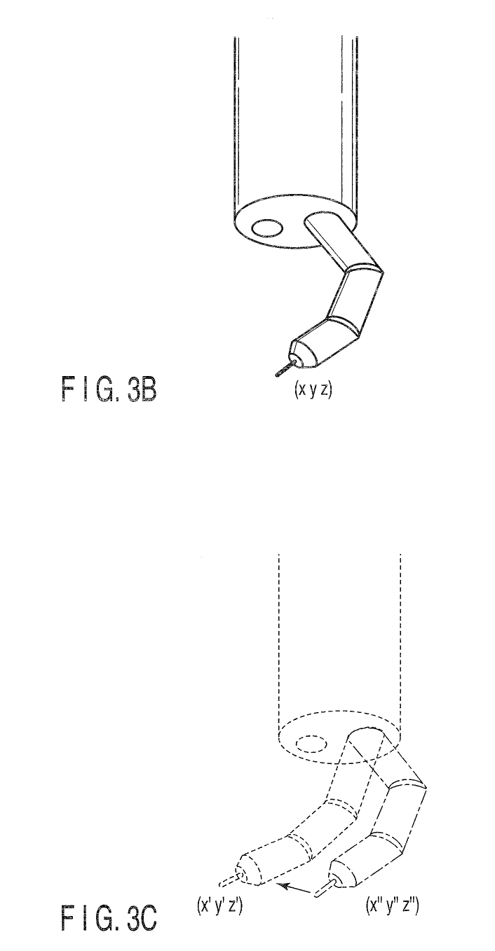

Then, the second estimation section of CPU 42 estimates the motion of the surgical section 21b based on the motion (the translation vector H and the rotation R) of the distal end of the endoscope (step S2). This motion will be described with reference to FIGS. 3A to (c). For the coordinates shown below, the origin (0,0,0) defines an X-axis, a Y-axis, and a Z-axis around the position of a view point set by the image pickup section 7 and optical system provided at the distal end of the endoscope.

As shown in FIG. 3A, it is assumed that for example, the surgical section 21b is located at the position (x,y,z) of the lesion 12 before the endoscope is moved for a certain reason. Then, the position (x′,y′,z′) taken by the surgical section 21b before the movement is expressed as follows:

$$( \begin{matrix}{x}^{′} \\ {y}^{′} \\ {z}^{′}\end{matrix} ) = {R}^{- 1} ( \begin{matrix}x - {H}_{x} \\ y - {H}_{y} \\ z - {H}_{z} \end{matrix} )$$

wherein H: translation of the endoscope and R: rotation of the endoscope. In contrast, the position (x″,y″,z″) taken by the surgical section 21b after the movement is expressed as follows.

$$( \begin{matrix}{x}^{″} \\ {y}^{″} \\ {z}^{″}\end{matrix} ) = ( \begin{matrix}x \\ y \\ z\end{matrix} )$$

At this time, the positional relationship between the distal end of the endoscope and the surgical arm section 21c (the portion extending from the forceps port 5 of the forceps channel) remains unchanged. Thus, the surgical section 21b has substantially moved from the position (x′,y′z′) to the position (x″,y″,z″).

The position (x,y,z) of the surgical section 21b is determined from the angle of each of the joints of the surgical arm section 21c determined from the rotational amount of the motor 31 moving the joint (the rotational amount is calculated from a value from the encoder connected to each of the motors 31), as well as the length of the joint and the connection status of the joint. However, the system needs to be initialized so that the motion of the surgical section 21b matches a coordinate system based on the distal end of the endoscope (for example, the system is initialized such that the UP/DOWN direction of bending of the endoscope matches the UP/DOWN direction of the surgical section 21b). Furthermore, the position (x′,y′,z′) taken by the surgical section 21b after the endoscope has been moved is calculated using Equations (1) and (2). From the resulting position (x′,y′,z′), joint length, and joint connection status, the angle of each joint can be determined based on reverse kinematics.

The reverse kinematics is a method of estimating a specific value for each joint (the angle of the joint or the like) from information on the position and posture of a manipulator (surgical instrument) in a work space. A joint parameter Φ for joints 1, 2, . . . , n is specified as follows.

Φ=(θ₁,θ₂, . . . , θₙ)^{T}

The position and posture of the manipulator are specified as follows.

Ep=(x_{Ep},y_{Ep},z_{Ep},Roll_{Ep},Yaw_{Ep},Pitch_{Ep})^{T}

Then, the relationship between the joint parameter and the position and posture of the manipulator is expressed by:

Eₚ=A(Φ)

Here, a target P for the position and posture of the manipulator is specified as follows.

Pp=(x_{Pp},y_{Pp},z_{Pp},Roll_{Pp},Yaw_{Pp},Pitch_{Pp})^{T}

Then, to set the manipulator in a condition indicated by Pₚ, Φ needs to be determined which satisfies:

Pₚ=A(Φ).

However, these relations are nonlinear. Thus, in order to find the value of Φ, a Jacobian determinant J(Φ) obtained by partially differentiating Ep by the elements of Φ is generally determined.

$$J ( Φ ) = ( \begin{matrix}\frac{d {x}_{ep}}{d {θ}_{1}} & \frac{d {x}_{ep}}{d {θ}_{2}} & … & \frac{d {x}_{ep}}{d {θ}_{n}} \\ \frac{d {y}_{ep}}{d {θ}_{1}} & \frac{d {y}_{ep}}{d {θ}_{2}} & … & \frac{d {y}_{ep}}{d {θ}_{n}} \\ \frac{d {z}_{ep}}{d {θ}_{1}} & \frac{d {z}_{ep}}{d {θ}_{2}} & … & \frac{d {z}_{ep}}{d {θ}_{n}} \\ \frac{d {Roll}_{ep}}{d {θ}_{1}} & \frac{d {Roll}_{ep}}{d {θ}_{2}} & … & \frac{d {Roll}_{ep}}{d {θ}_{n}} \\ \frac{d {Yaw}_{ep}}{d {θ}_{1}} & \frac{d {Yaw}_{ep}}{d {θ}_{2}} & … & \frac{d {Yaw}_{ep}}{d {θ}_{n}} \\ \frac{d {Pitch}_{ep}}{d {θ}_{1}} & \frac{d {Pitch}_{ep}}{d {θ}_{2}} & … & \frac{d {Pitch}_{ep}}{d {θ}_{n } }\end{matrix} )$$

Then, Φ satisfying Pₚ=A(Φ) is determined from Equation 9 by convergence calculation.

\dot{Φ}=J(Φ)⁻¹Ėₚ

from

Pₚ=A(Φ)

Based on the calculated operation amount, the joints of the surgical arm section 21c are bent to move the surgical section 21b to the position of the lesion 12 (step S4).

The surgical arm section 21c is bent, rotated, and moved forward and backward so as to hold the position of the surgical section 21b. This is repeated until the surgical section 21b completes the surgery on the lesion 12 (step S5).

That is, an inverse problem is used to determine the required bending, rotation, or forward or backward movement so that the required movement can be performed.

As described above, according to the present embodiment, even if during surgery with the surgical instrument, the surgeon moves the observation visual field for the surgeon's own convenience or biological motion in the body cavity moves the endoscope, the surgical section of the surgical instrument is prevented from being moved away from the target site being operated on. The position of the surgical section thus remains unchanged. This eliminates the need for the surgeon's moving operation of returning the surgical section to the original position. Thus, the surgeon can concentrate on the surgical procedure and have the burden of the manipulation and fatigue reduced.

Now, a second embodiment of the present invention will be described.

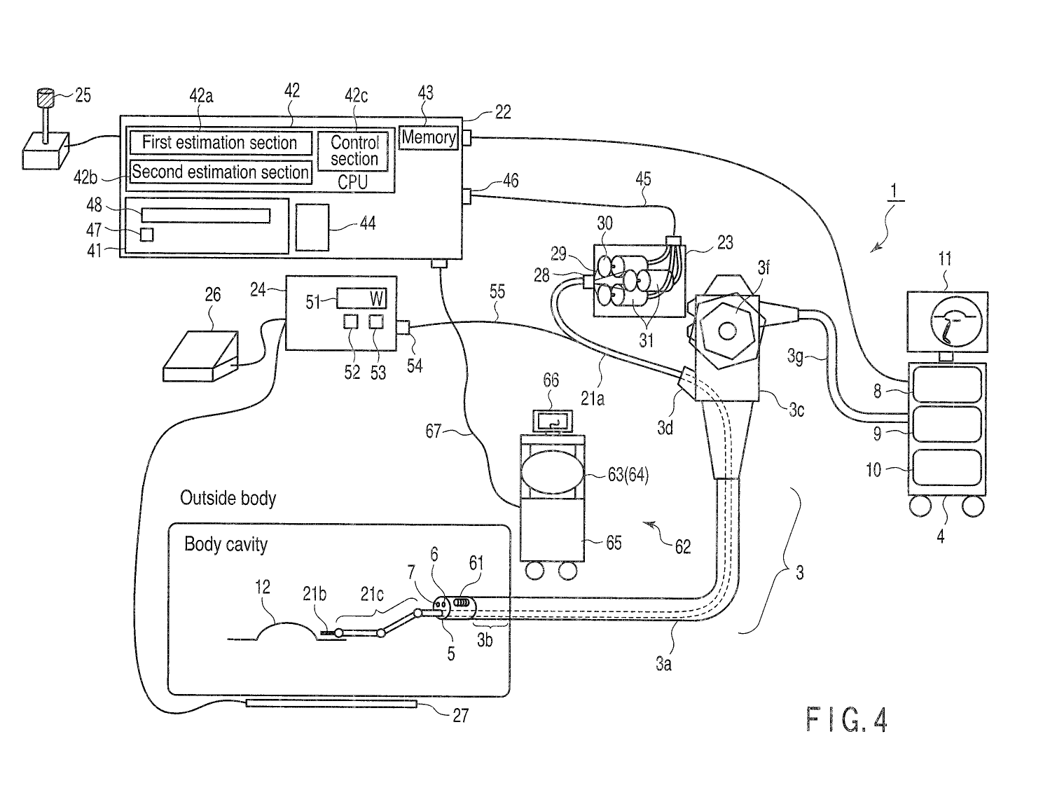

FIG. 4 is a diagram showing the general configuration of an endoscopic system in which a surgical instrument position control apparatus according to the present embodiment is mounted.

The endoscopic system corresponds to the configuration of the endoscopic system according to the first embodiment shown in FIG. 1 described above and in which a magnetic field generation coil is provided on the distal end side of the endoscope main body 3. In the present endoscopic system, a magnetic field generated by the magnetic field generation coil is detected by an endoscope shape observation device to estimate the position of the distal end of the endoscope. In the present embodiment, the components other than the magnetic field generation coil and the endoscope shape observation device are equivalent to the corresponding ones in the endoscopic system shown in FIG. 1. Thus, these components are denoted by the same reference numerals and will not be described below.

As shown in FIG. 4, the endoscope main body 3 includes at least one magnetic field generation coil 61 at a position closer to the distal end of the insertion section 3a than the bending section 3b. Furthermore, an endoscope shape observation device 62 is located near the patient. The endoscope shape observation device 62 is composed of a magnetic field detection unit 63, a position estimation section 65, and a monitor 66.

The magnetic field detection unit 63 includes a plurality of magnetic field detection coils 64 (64a, 64b, . . . , 64n) arranged inside a housing to detect a magnetic field generated by the magnetic field generation coil 61. The position estimation section 65 receives data on the magnetic field detected by the magnetic field detection coil 64 to estimate the position of the magnetic field generation coil 61. The estimated position of the magnetic field generation coil 61 is a value in a coordinate system based on the magnetic field detection unit 63. This is disclosed in Japanese Patent No. 3571675 in detail. Estimated positional information on the magnetic field generation coil 61 is transmitted to the surgical instrument control section 22 through a signal line 67. CPU 42 in the surgical instrument control section 22 calculates the position of the distal end of the insertion section 3a (the view point position set based on the image pickup element and the optical system) the image of which is taken by the image pickup section 7, based on the positional relationship (design value) with the magnetic field generation coil 61. CPU 42 then calculates the amount of movement (the magnitude of the corresponding translation vector) between images based on the calculated position of the distal end of the insertion section 3a. CPU 42 then allows the first estimation section 42a to estimate the moving direction and amount of the insertion section 3a of the endoscope using the calculated moving amount (the magnitude of the translation vector).

In the present configuration example, the magnetic field detection coil 64 is provided inside the housing of the magnetic field detection unit 63. However, the magnetic field detection coil 64 may be located near and around the patient.

According to the endoscopic system in which the surgical instrument position control apparatus according to the second embodiment described above is mounted, the motion amount (the magnitude of the translation vector) of the endoscope can be accurately calculated. Thus, the surgical section 2c can be accurately moved.

Now, a third embodiment of the present invention will be described.

In the third embodiment, the movement of the distal end of the endoscope is estimated based on the matching between images to determine the position of the surgical instrument. Then, the surgical instrument is moved parallel to the image pickup surface of the image pickup element back to the original position. The configuration of the present embodiment is the same as that of the first embodiment as shown in FIG. 1.

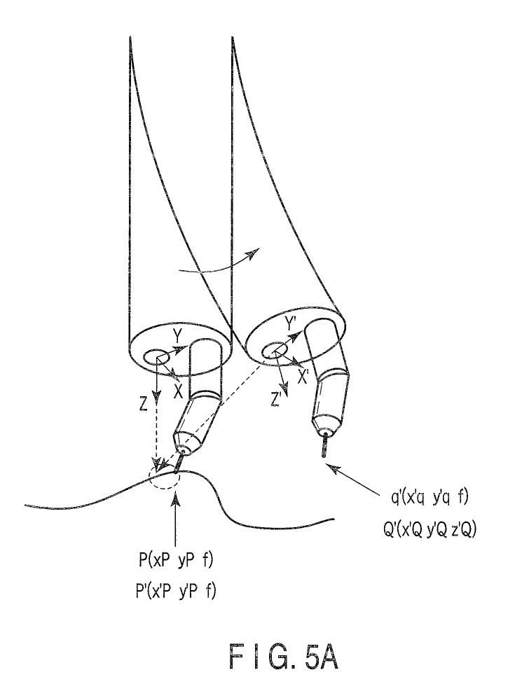

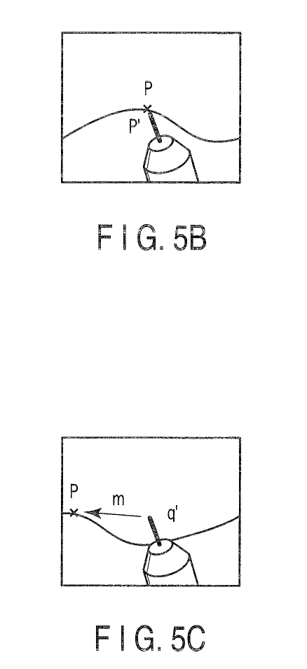

As shown in FIGS. 5A, 5B, and 5C, p(xp,yp,f) [f: the focal distance of the optical system in the image pickup section 7] denotes the position of the distal end (the surgical section 21b or lesion 12) of the surgical instrument 21 in the observation visual field, on the screen obtained before movement, of the endoscope being used to operate on the lesion. P′(xp′,yp′,zp′) denotes the position of the surgical procedure after the endoscope has been moved (the position taken by the surgical instrument 21 before the endoscope has been moved). In an image taken after the endoscope has been moved, the position (p) taken by the surgical section 21 before the movement can be determined based on the matching between images. The matching is a process of using, as a template, a part of an image taken before the movement which corresponds to the vicinity of the position (p) where the distal end of the surgical section 21 is present, to search an image taken after the movement of the endoscope, thus determining the position. If the surgical instrument 21 is present in the template, the region of the surgical section 21 is detected and excluded from a target for a process of calculating the level of the matching between the images. The movement, on the screen, from the position q′(xq′,yq′,f) taken by the distal end of the surgical section 21 after the movement of the endoscope to the position p′(xp′,yp′ f) of the surgical procedure observed after the movement of the endoscope is as shown in:

$$m = ( \begin{matrix}{x}_{q}^{′} - {x}_{p}^{′} \\ {y}_{q }^{′} - {y}_{p}^{′} \\ 0\end{matrix} )$$

where f denotes the focal distance of the image pickup section 7 incorporated into the tip of the endoscope.

For the three-dimensional position Q′(XQ′,YQ′ZQ′) of the distal end of the surgical section 21, the magnitudes of the bending, rotation, and forward and backward movements of each of the joints of the surgical section 21 are determined from values from the encoder connected to the respective motors. If the value ZQ′ of the Z component of the distal end of the surgical section 21 obtained after the movement of the endoscope is used to move the distal end of the surgical section 21 parallel to the image pickup surface of the image pickup element of the image pickup section 7 to the vicinity of the position of the surgical procedure (the position of the lesion 12: the position where the distal end of the surgical section 21 is present before the movement of the endoscope), the direction and magnitude of the movement are expressed as shown in:

$$M = \frac{{z}_{q}^{′}}{f} ( \begin{matrix}\begin{matrix}{x}_{q}^{′} - {x}_{p}^{′} \\ {y}_{q}^{′} - {y}_{p}^{′}\end{matrix} \\ f\end{matrix} ) .$$

As described above, according to the endoscopic system in which the surgical instrument position control apparatus according to the present embodiment is mounted, if the endoscope is moved parallel to the image pickup surface of the image pickup element, the motion of the endoscope can be estimated more quickly than in the first and second embodiments. Thus, the surgical section can be moved at a speed close to that in real time.

Now, a fourth embodiment of the present invention will be described.

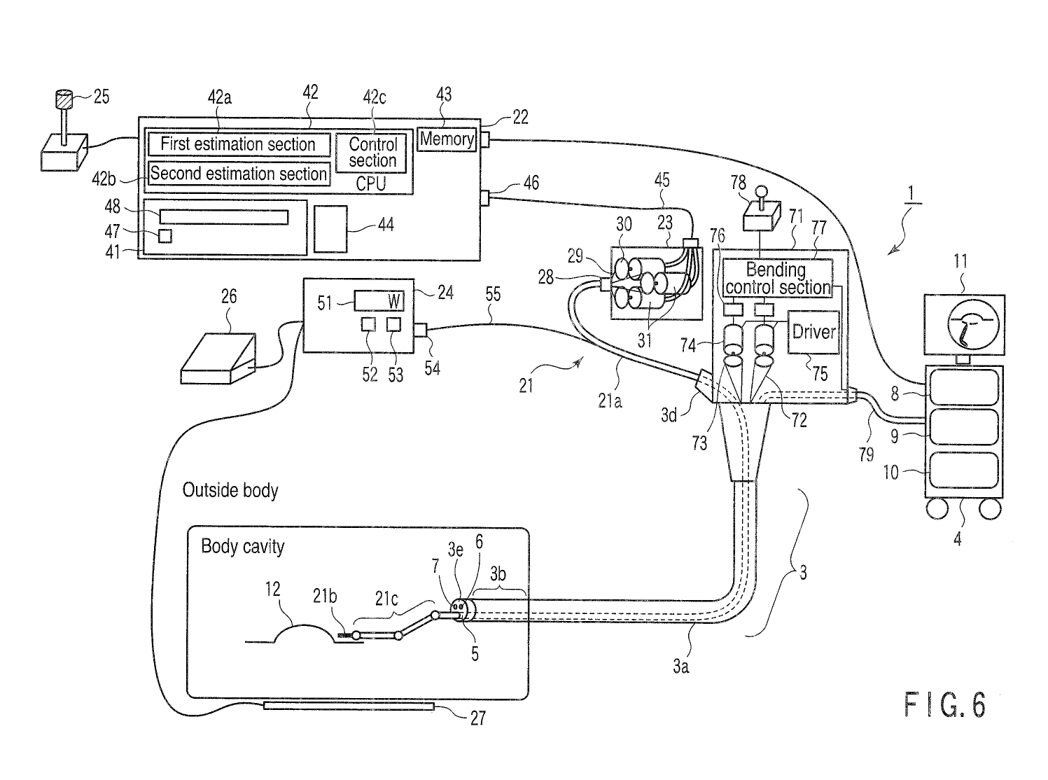

The endoscope according to the fourth embodiment of the present invention shown in FIG. 6 is configured such that the bending section of the endoscope main body is electrically bent. In the present embodiment, the endoscope main body is electrically driven, and the other components are equivalent to the corresponding ones in the first embodiment shown in FIG. 1 described above. Thus, these components are denoted by the same reference numerals and will not be described below.

An electrically-operated bending manipulation section 71 in the endoscope main body 3 includes a plurality of wires 72 one end of each of which is connected to the bending section 3b, a plurality of pulleys 73 each coupled to the other end of the corresponding wire 72, motors 74 each including a rotating shaft on which the corresponding pulley 73 is fittingly installed, a driver 75 individually driving the respective motors 74, encoders 76 each provided in the corresponding motor, and a bending control section 77 controlling the motor driver 75 based on values detected by the encoders 76. Moreover, the bending control section 77 is connected to a bending joystick 78 used to specify a bending operation.

Furthermore, the electrically-operated bending manipulation section 71 is connected to the apparatus main body 4 via a cable 79. The cable 79 includes a light guide fiber through which illumination light is transmitted, and signal lines including an image signal line and a control signal line. Furthermore, in the configuration example described in the present embodiment, the joystick is provided in each of the endoscope and the surgical instrument. However, these manipulation functions are collectively provided in one joystick. Moreover, the present embodiment may include the magnetic field generation coil and endoscope shape observation device provided in the endoscopic system according to the second embodiment.

The amounts by which the endoscope main body and the surgical instrument are bent are determined from the amounts (encoder output values) by which the motors pulling the respective wires are rotated. Thus, the motion of the distal end of the endoscope is estimated from the rotational amounts of the endoscope motors. The distal end of the surgical instrument is moved based on the estimated values.

According to the endoscopic system in which the surgical instrument position control apparatus according to the present embodiment is mounted as described above, not only the effects of the first and third embodiments are exerted but also the bending operations of the surgical instrument and the endoscope are specified using the manipulation switch made up of a joystick or the like and which can be easily manipulated. This enables a reduction in the surgeon's fatigue and burden. Moreover, when the present embodiment includes the magnetic field generation coil and endoscope shape observation device in the endoscopic system according to the second embodiment, the position detection based on image processing is avoided, eliminating the need for advanced calculations by CPU. Thus, the motion of the distal end of the endoscope can be easily estimated, enabling the distal end (surgical section) of the surgical instrument to be moved at a speed close that in real time. Furthermore, the motion amount (the magnitude of the translation vector) of the endoscope can be accurately calculated, allowing the surgical section 2c to be accurately moved.

In the configuration example in the present embodiment, the electrically-operated endoscope main body is used. However, even a non-electrically-operated, normal endoscope main body may be used to exert similar effects by providing a sensor at the insertion port 3d of the forceps channel or the forceps port 5 to detect the traction amount of the wire, and providing a mechanism detecting the rotational amount of each manipulation dial 3f installed in the manipulation section to determine the traction amount of the wire.

Now, a fifth embodiment of the present invention will be described.

In an endoscopic system in which a surgical instrument position control apparatus according to the present embodiment is mounted, if the movement amount of the endoscope is smaller then a preset threshold value, the position of the surgical instrument is maintained without change. This is to prevent a possible situation where if the distal end of the surgical section is always moved in conjunction with movement of the endoscope, the distal end of the surgical instrument is always vibrated by noise, calculation errors, or the like, making the surgical procedure difficult. Software (program) can be configured to provide a threshold for the movement amount of the endoscope according to the present embodiment. Thus, the above-described arrangement is applicable to the configuration of any of the above-described first to fourth embodiments.

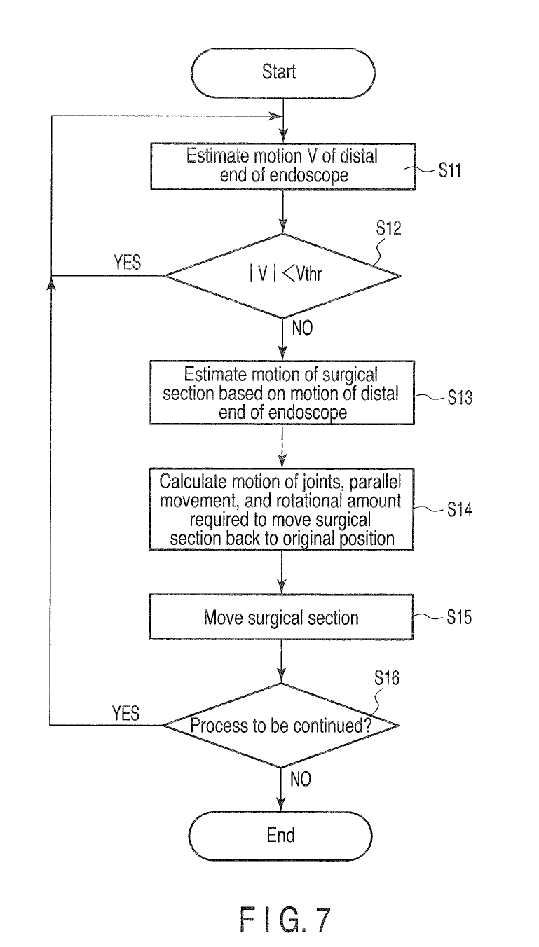

This moving process will be described in detail with reference to the flowchart shown in FIG. 7. In this case, an example in which the moving process is applied to the endoscopic system according to the first embodiment will be described.

First, the motion vector of the endoscope main body 3 with respect to the target site is estimated based on time-sequentially adjacent images obtained (step S11). Specifically, inter-image shift maps are created based on time-sequentially consecutively input image data obtained by the image pickup section 7. For an optionally specified target site (for example, the lesion 12) in the screens, one inter-image shift map corresponds to estimation of one inter-image motion vector (a translation vector (h) and rotation matrix R: the translation vector is a unit vector). The motion vector, that is, the motion (relative motion) of the distal end of the endoscope, is estimated based on the shift map corresponding to each image. The magnitude of the translation vector is then determined to calculate the absolute motion V of the distal end of the endoscope.

The calculated absolute value |V| of the motion of the distal end of the endoscope is compared with an empirically or experimentally determined threshold Vthr (step S12). In the comparison, if the motion |V| of the distal end of the endoscope is smaller than the threshold Vthr (YES), the device determines that this level of movement of the endoscope does not cause the process of moving the surgical instrument to be executed. The device returns to step S11 to continuously estimate the motion of the distal end of the endoscope. On the other hand, if the motion |V| is larger than the threshold Vthr (NO), the device determines that the process of moving the surgical instrument is to be executed. The device shifts to step S13. Step S13 corresponds to step S2 in FIG. 2 described in the first embodiment. The motion of the surgical section 21b is estimated based on the motion (the translation vector H and the rotation R: see FIG. 3) of the distal end of the endoscope (step S13). As shown in FIG. 3, the device determines, for example, the position (x,y,z) of the surgical section 21b to be moved by the endoscope and the position (x′,y′z′) taken by the surgical section 21b after the movement of the endoscope and before the surgical section 21b is positionally corrected. In connection with this, the position (x″,y″,z″) of the moved surgical section 21b is determined. Then, based on the calculated positions of the surgical section 21b, the device calculates the amount of movement from the coordinate position (x″,y″,z″) taken by the surgical section 21b after the movement of the endoscope to the position (x′,y′,z′), that is, the amounts of operations, such as bending, rotation, and forward and backward movements, of the joints of the surgical arm section 21c which amounts are required to move the surgical section 21b to the original position of the lesion 12 (step S14).

Then, based on the calculated operation amounts, the joints of the surgical arm section 21c are bent to move the surgical section 21b to the position of the lesion 12 (step S15). The surgical arm section 21c is bent, rotated, and moved forward and backward so as to hold the position of the surgical section 21b. This is repeated until the surgical section 21b completes the surgery on the lesion 12 (step S16).

As described above, according to the present embodiment, the particular threshold is provided for the motion of the endoscope. This prevents a possible situation where the distal end of the surgical instrument is always vibrated by noise, calculation errors, or the like, making the surgical procedure difficult. Thus, when the surgeon operates on the patient, possible unwanted vibration is prevented. Consequently, the surgeon can concentrate on the surgical procedure and have the burden of the manipulation and fatigue reduced.

Even if the surgeon moves the observation visual field for the surgeon' own convenience or biological motion in the body cavity moves the endoscope, the surgical section of the surgical instrument is prevented from being moved away from the target site being operated on. The position of the surgical section thus remains unchanged.

The present invention can provide the surgical procedure position control apparatus for the endoscope which, even when the distal end of the endoscope is moved during surgery with the surgical instrument, allows the surgical instrument to be prevented from being moved away from the site being operated on, enabling the surgical procedure to be continued.

When the endoscope is moved in response to the surgeon's manipulation or biological motion in the body cavity, the surgical instrument position control apparatus estimates the motion of the distal end of the insertion section of the endoscope with respect to the target site. Based on the motion of the distal end of the insertion portion, the surgical instrument position control apparatus further estimates the motion of the surgical instrument. Moreover, the surgical instrument position control apparatus calculates the amounts of operations, such as bending, rotation, and forward and backward movements, of the joints located at the distal end of the surgical instrument which amounts are required to move the surgical section from the position taken by the surgical instrument after the movement of the endoscope to the surgical procedure position. The surgical instrument position control apparatus can thus operate the joints to move the distal end of the surgical instrument to the original position so as to hold the position of the surgical instrument every time the endoscope is moved. This enables a reduction in the burden of the manipulation and fatigue.

Furthermore, the surgical instrument position control apparatus includes the magnetic field generation coil provided at the distal end of the endoscope. The surgical instrument position control apparatus estimates the position and motion of the distal end of the endoscope based on a generated magnetic field, to estimate the motion of the surgical instrument based on the motion of the distal end. Thus, the surgical instrument position control apparatus can thus quickly calculate the operation amounts required to move to the original surgical procedure position and return the surgical instrument to the surgical procedure position. Moreover, the threshold for the detection of movement of the endoscope is provided. This prevents possible vibration associated with the unwanted position holding operation for the surgical instrument performed as a result of noise, calculation errors, or the like. Therefore, the surgical procedure is prevented from being made difficult.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A surgical instrument position control apparatus for an endoscope allowing a biomedical tissue in a body cavity to be observed comprising:
a surgical instrument having a surgical section which is movable forward and backward through an insertion section of the endoscope and which allows the biomedical tissue to be operated on, and a movable section to bend and move forward and backward the surgical section;
a driving mechanism bending and moving forward and backward the movable section of the surgical section to move a position of the surgical section;
an estimation section estimating a moving direction and a moving amount of the surgical section in conjunction with movement of a distal end of the insertion section; and
a control section drivingly controlling the driving mechanism based on the moving direction and amount of the surgical section so that the surgical section is returned to a position taken by the surgical section before the endoscope is moved.

2. The surgical instrument position control apparatus for the endoscope according to claim 1, wherein the movable section of the surgical section has an articulated structure including a plurality of joints and rods connecting the joints together.

3. The surgical instrument position control apparatus for the endoscope according to claim 2, wherein the estimation section includes:
a first estimation section estimating the motion of the endoscope; and
a second estimation section estimating the moving direction and amount of the surgical section of the surgical instrument with respect to the motion of the endoscope, based on a preset positional relationship between the endoscope and the surgical instrument,
wherein a position of the surgical section is estimated based on the motion of the surgical section on an image picked up by an image pickup section provided at a distal end of the insertion section of the endoscope, angle information on the joints of the movable section of the surgical instrument which information is loaded into the control section, and a status of the surgical instrument based on estimated translation movement information and rotational information.

4. The surgical instrument position control apparatus for the endoscope according to claim 3, wherein the driving mechanism comprises:
a plurality of wires one end of each of which is connected to a corresponding one of the joints of the movable section; and
an electrically-operated driving mechanism controlled by the control section and connected to another end of each of the wires to pull and loosen any of the wires to bend the movable section in a desired direction,
wherein based on the moving direction and amount of the surgical section determined based on the position estimated by the estimation section, the control section drives the electrically-operated driving mechanism to return the surgical instrument to the position taken by the surgical section before the endoscope is moved.

5. A surgical instrument position control apparatus for an endoscope allowing a biomedical tissue in a body cavity to be observed comprising:
a surgical instrument having a surgical section which is movable forward and backward through an insertion section of the endoscope and which allows the biomedical tissue to be operated on, and a movable section to bend and move forward and backward the surgical section;
a driving mechanism bending and moving forward and backward the movable section of the surgical section to move a position of the surgical section;
a magnetic field generation section provided in the insertion section of the endoscope;
an estimation section detecting a magnetic field generated by the magnetic field generation section, determining a position of the surgical section from a position of the magnetic field generation section based on the detected magnetic field, and estimating a moving direction and a moving amount of the surgical section in conjunction with movement of a distal end of the insertion section; and
a control section drivingly controlling the driving mechanism based on the moving direction and amount of the surgical section so that the surgical section is returned to a position taken by the surgical section before the endoscope is moved.

6. The surgical instrument position control apparatus for the endoscope according to claim 1 or 5, wherein upon estimating that the movement of the endoscope is parallel to an image pickup surface of an image pickup section provided at a distal end side of the insertion section, the estimation section of the surgical instrument fixes a distance in a depth direction orthogonal to the image pickup surface, to a focal direction of an optical system in the image pickup section, to estimate the moving direction and amount of the surgical section.

7. The surgical instrument position control apparatus for the endoscope according to claim 1 or 5, wherein the surgical instrument is inserted through the insertion section of the endoscope so as to be movable forward and backward, the insertion section having an electrically-operated driving mechanism bending, in a desired direction, a bending section provided at a distal end of the insertion section of the endoscope.

8. The surgical instrument position control apparatus for the endoscope according to claim 1 or 5, wherein the estimation section holds a position of the surgical instrument if the moving amount of the insertion section of the endoscope is smaller than a preset threshold.

9. The surgical instrument position control apparatus for the endoscope according to claim 1 or 5, wherein for an optionally specified target site in a screen in images picked up by the image pickup section, the control section estimates motion of the distal end of the endoscope between time-sequentially adjacent images to determine magnitude of a translation vector in the motion to calculate absolute motion of the distal end of the endoscope.

10. The surgical instrument position control apparatus for the endoscope according to claim 1 or 5, wherein the insertion section of the endoscope has an electrically-operated driving mechanism comprising an articulated structure having a plurality of joints each of which is bent by pulling and loosening a wire coupled to a pulley attached to a shaft of a motor, the electrically-operated driving mechanism being driven by the motor in accordance with an instruction from the control section to perform a moving operation and a posture holding operation.

11. The surgical instrument position control apparatus for the endoscope according to claim 4, wherein the electrically-operated driving mechanism for the insertion section of the endoscope and the electrically-operated driving mechanism for the movable section of the surgical section are master slave type devices each including an input device manipulated by a surgeon to remotely specify positions and postures of the insertion section and the surgical section.

12. The surgical instrument position control apparatus for the endoscope according to claim 4, wherein the estimation section estimates the moving amount of the surgical section based on operation amounts of bending, rotation, and forward and backward movements of each of the joints.

13. The surgical instrument position control apparatus for the endoscope according to claim 10, wherein the electrically-operated driving mechanism for the insertion section of the endoscope and the electrically-operated driving mechanism for the movable section of the surgical section are master slave type devices each including an input device manipulated by a surgeon to remotely specify positions and postures of the insertion section and the surgical section.

14. The surgical instrument position control apparatus for the endoscope according to claim 10, wherein the estimation section estimates the moving amount of the surgical section based on operation amounts of bending, rotation, and forward and backward movements of each of the joints.
